(19) United States
(12) Patent Application Publication
(10) Pub. No.: US 2018/0001054 A1
(43) Pub. Date: Jan. 4, 2018

(54) CATHETER SYSTEMS AND ENDOSCOPIC SYSTEMS WITH REDUCED BACKLASH

(71) Applicant: Jacek KRZYZANOWSKI, Etobicoke (CA)

(72) Inventor: Jacek KRZYZANOWSKI, Etobicoke (CA)

(21) Appl. No.: 15/543,353

(22) PCT Filed: Jan. 12, 2016
(86) PCT No.: PCT/CA2016/050026

§ 371 Date: Jul. 13, 2017

Related U.S. Application Data
(60) Provisional application No. 62/104,905, filed on Jan. 19, 2015.

(51) Int. Cl. A61M 25/00; A61B 1/005; A61B 1/018; A61M 25/09

(52) U.S. Cl. CPC A61M 25/0043; A61M 25/09; A61B 1/0051; A61B 1/018

(57) ABSTRACT

Medical instruments such as catheter systems and endoscopic systems that exhibit reduced backlash during their operation are disclosed. The reduction of backlash avoids the use of protrusions or fins that can significantly obstruct fluid flow through working channels of the instruments. According to one embodiment, a medical instrument comprises a first member having an inside surface defining a first lumen and a second member extending in the first lumen and having an outside surface cooperating with the inner surface of the first member to define an intermediate longitudinal passage therebetween. One of the inside surface of the first member and the outside surface of the second member is permanently formed to have a sinuous shape defining predisposed locations for contacting the other one of the inner surface of the first member and the outer surface of the second member to reduce backlash during relative longitudinal movement.

### CROSS-REFERENCE TO RELATED APPLICATION(S)

The present application claims priority to U.S. provisional patent application No. 62/104,905 filed on Jan. 19, 2015, the entire contents of which are hereby incorporated by reference.

### TECHNICAL FIELD

The disclosure relates generally to medical instruments such as catheter and endoscopic systems, and more particularly to reducing backlash in such instruments.

### BACKGROUND OF THE ART

Medical instruments such as catheters, endoscopic instruments, and, endoscopic and catheter systems are usually constructed of long tubular or tubular-like main bodies connecting opposite ends of devices useful for carrying out medical procedures. Endoscopes can include an insertion tube, defining one or more lumens therethrough where at least one of which is a working channel that receives an endoscopic instrument for procedural purposes. Endoscopes incorporate optical or electronic imaging systems for the transmission of images from bodily cavities for the visualization of the tissue. Endoscopes or parts thereof can be rigid or flexible. Working channels can be located on the outside or inside of the insertion tube of an endoscope. Some of today's sophisticated catheters can resemble endoscopes without the imaging system.

The proximal ends of catheters and endoscopic instruments are typically associated with some actuating means and the distal ends are typically associated with end effectors configured to deliver a desired action to a treatment site. Some endoscopic and catheter systems incorporate an outer tubular member and an inner member insertable therethrough. The mechanical actuation of the end effectors is usually achieved by a relative longitudinal movement between the outer tubular member and the inner member via the action of the actuating means. Actuation can comprise pushing and/or pulling of one of the tubular member and inner member relative to the other to permit, for example, the opening and closing of forceps. Alternatively, a biopsy or other needle coupled to the inner member may be inserted into tissue of a patient by movement of the inner member relative to the outer tubular member.

In such instruments, an outside diameter of the inner member may be smaller than an inside diameter of the tubular member to provide an annular space between the inner member and the tubular member to permit passage of fluid during irrigation or suctioning of the treatment site. The space provided between the inner member and the tubular member can be a cause backlash (i.e., lost motion) experienced by the operator when relative movement between the inner member and the tubular member is carried out to operate the instrument. The amount of backlash can be significant depending on the length and configuration of the instrument and whether the instrument is bent to accommodate the anatomy of the patient during a procedure.

Improvement is therefore desirable.

### SUMMARY

In one aspect, the disclosure describes a medical instrument comprising:

a first elongate member having an inside surface defining a first lumen having a first center line; and

a second elongate member extending in the first lumen and having an outside surface cooperating with the inside surface of the first member to define a first intermediate longitudinal passage therebetween extending in the first lumen, the second member having a second center line;

wherein the first member and the second member are permanently formed to have non-parallel portions between the first center line and the second center line and define one or more predisposed contact locations between the inside surface of the first member and the outside surface of the second member to reduce backlash during relative longitudinal movement between the first member and the second member.

In another aspect, the disclosure describes a medical instrument comprising:

a first member having an inside surface defining a first lumen; and

a second member extending in the first lumen and having an outside surface cooperating with the inner surface of the first member to define an intermediate longitudinal passage therebetween extending in the first lumen;

wherein one of the inside surface of the first member and the outside surface of the second member is permanently formed to have a sinuous shape defining one or more predisposed locations for contacting the other one of the inner surface of the first member and the outer surface of the second member to reduce backlash during relative longitudinal movement between the first member and the second member.

In a further aspect, the disclosure describes a medical instrument comprising:

a first member having an inside surface defining a first lumen having a first center line, the inside surface being defined by a first cross-sectional profile swept along the first center line; and

a second member extending in the first lumen and having an outside surface cooperating with the inner surface of the first member to define a first intermediate longitudinal passage therebetween extending in the first lumen, the second member having a second center line, the outside surface of the second member being defined by a second cross-sectional profile swept along the second center line;

wherein one of the first member and the second member is permanently formed to have to have a sinuous respective first center line or second center line and define one or more predisposed locations for contacting the other one of the inside surface of the first member and the outer surface of the second member to reduce backlash during relative longitudinal movement between the first member and the second member.

Further details of these and other aspects of the subject matter of this application will be apparent from the detailed description and drawings included below.

### DESCRIPTION OF THE DRAWINGS

Reference is now made to the accompanying drawings, in which:

FIGS. 1A-1D illustrate different schematic views of an exemplary medical instrument according to one embodiment of the present disclosure;

FIGS. 2A-2C illustrate different schematic views of an exemplary medical instrument according to another embodiment of the present disclosure;

FIGS. 3A-3C illustrate different schematic views of an exemplary medical instrument according to another embodiment of the present disclosure;

FIGS. 4A and 4B illustrate different schematic views of an exemplary medical instrument according to another embodiment of the present disclosure;

FIGS. 5A and 5B illustrate different schematic views of an exemplary medical instrument according to another embodiment of the present disclosure;

FIGS. 6A and 6B illustrate different schematic views of an exemplary medical instrument according to another embodiment of the present disclosure; and

FIGS. 7A and 7B illustrate different schematic views of an exemplary medical instrument according to another embodiment of the present disclosure.

### DETAILED DESCRIPTION

The present disclosure relates to medical instruments such as catheter and endoscopic systems that exhibit reduced backlash during their operation. In various embodiments, the reduction of backlash avoids the use of protrusions or fins hat can significantly obstruct fluid flow through working channels of the instruments. Aspects of various embodiments are described through reference to the drawings.

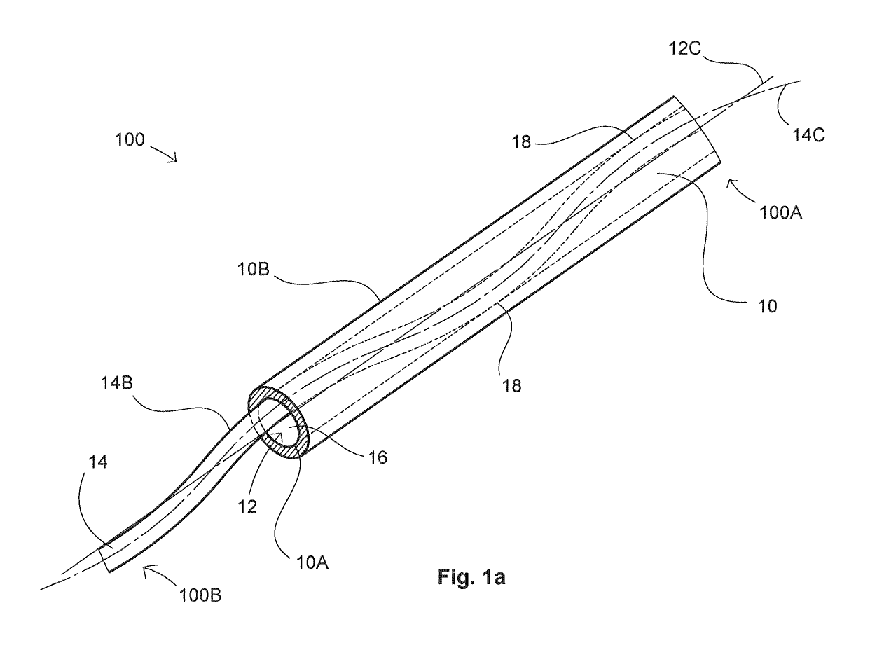

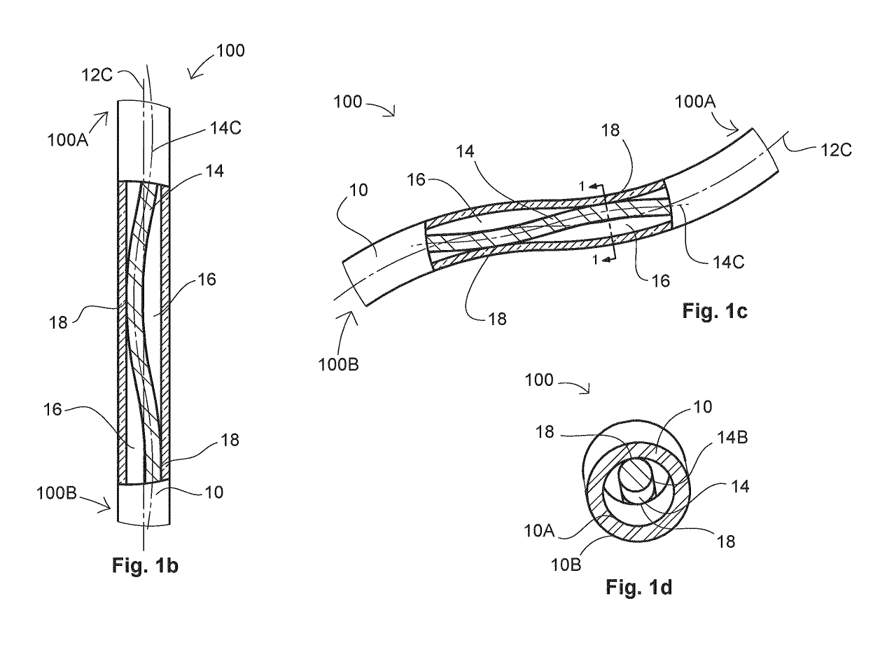

FIGS. 1A-1D illustrate different views of an exemplary medical instrument 100 according to one embodiment of the present disclosure. Medical instrument 100 may comprise or be part of a catheter or an endoscopic system for example. Accordingly, medical instrument 100 may have a generally elongated shape. Medical instrument 100 may have proximal portion 100A that may be associated with one or more actuation means (not shown) of known or other type(s) and distal portion 100B that may be associated with one or more end effectors (not shown) of known or other type(s).

FIG. 1A shows a perspective view of medical instrument 100. Medical instrument 100 may comprise first (e.g., outer) elongate member 10 having inside surface 10A and outside surface 10B. First member 10 may have a generally tubular configuration. First member 10 may be made of flexible or rigid material(s) typically used in the construction of catheter or endoscopic systems. Inside surface 10A of first member 10 may define lumen 12 (e.g., working channel for an endoscopic instrument) extending through first member 10. Lumen 12 may have center line 12C. Lumen 12 may have a substantially uniform cross-sectional profile along center line 12C where the cross-sectional profile is substantially perpendicular to center line 12C. Alternatively, lumen 12 may have a non-uniform cross-sectional profile along center line 12C. The cross-sectional profile of lumen 12 may be substantially circular and smooth or may be of other suitable shape. For example, inside surface 10A defining lumen 12 may itself be defined by a cross-sectional profile having been swept along center line 12C. A substantially circular cross-sectional profile of lumen 12 may be advantageous in some cases to facilitate a sealed connection between lumen 12 and some other fluid conduit(s) or device(s) that may be required during use. For example, the substantially smooth and circular cross-sectional profile of lumen 12 may facilitate sealing with endoscopic accessories such as pumps, light sources, syringes, electrical connectors, optical connectors, valves, handles, actuators, luerlocks, tubing, sheaths, needles, O-rings, gaskets, eyelets, ferrules, etc.

Medical instrument 100 may comprise second (e.g., inner) elongate member 14 (e.g. endoscopic instrument) extending substantially longitudinally in lumen 12 and having outside surface 14B. Second member 14 may extend entirely or only partially in lumen 12. Second member 14 may be longitudinally movable relative to first member 10 during operation of medical instrument 10. Alternatively, first member 10 may be longitudinally movable relative to second member 14 during operation of medical instrument 100. The relative movement between first member 10 and second member 14 may serve to actuate an end effector that may be connected to or that may be part of medical instrument 100.

Second member 14 may have center line 14C. Second member 14 may have a tubular (e.g., hollow cross-section) or solid configuration. For example, second member 14 may comprise a guide wire or other endoscopic instrument that may be pushed or pulled relative to first member 10. In various embodiments, second member 14 may be constructed of multiple strands, have a lubricious coating thereon, have an insulating coating thereon, have a core and cladding construction, have different outer diameters and/or be tapered. Second member 14 may have a substantially uniform (e.g., outer) cross-sectional profile along center line 14C where the cross-sectional profile is substantially perpendicular to second center line 14C. Alternatively, second member 14 may have a non-uniform cross-sectional profile along center line 14C. The cross-sectional profile of second member 14 may be substantially circular and smooth or may be of other suitable shape. For example, outside surface 14B of second member 14 may itself be defined by a cross-sectional profile having been swept along center line 14C. A substantially circular cross-sectional profile of second member 14 may be advantageous in some cases to facilitate a sealed connection between second member 14 and some other fluid conduit(s) or device(s) as listed above that may be required during use.

Second member 14 may have a smaller outer cross-sectional dimension (e.g., diameter) than the cross-sectional dimension (e.g., diameter) of lumen 12. Accordingly, outside surface 14B of second member 14 may cooperate with inside surface 10A of first member 10 to define an intermediate longitudinal passage 16 therebetween extending longitudinally in lumen 12. Passage 16 may be configured to permit the passage of fluid though lumen 12 during irrigation or suction of a treatment site for example.

The dimensions of catheters, endoscopic devices and systems are typically determined and limited based on the human anatomy or the animal anatomy in veterinary procedures. For example, inside diameters of microcatheters for neurological use can be as small as 0.017″ (0.43 mm) in diameter and vascular catheters may have inside diameters as small as 0.021″ (0.53 mm). Guide wires (e.g., second member 14) that are used with microcatheters may be as small as 0.014″ (0.36 mm) in diameter. Inside diameters of working channels (e.g., lumen 12) of flexible endoscopes (e.g., first member 10) typically vary from 2 to 4.2 mm and the outside diameters of endoscopic instruments (e.g., second member 14), such as snares, guide wires, endoscopic retrograde cholangiopancreatography (ERCP) devices are limited by the inside diameters of the working channels (e.g., lumen 12) of flexible endoscopes.

In various embodiments, first member 10 and second member 14 may be permanently formed to have non-parallel portions between center line 12C of lumen 12 and center line 14C of second member 14. The non-parallel potions may define one or more predisposed contact locations 18 between inside surface 10 of first member 10 and outside surface 14B of second member 14. For example, such non-parallel portions may include adjacent portions of center line 12C and center line 14C of differing slopes. The non-parallel portions between center line 12C and center line 14C may cause inside surface 10A and outside surface 14B to also have non-parallel portions. Predisposed contact locations 18 may contribute toward reducing backlash during relative longitudinal movement between first member 10 and the second member 14.

Depending on the specific configuration and state of medical instrument 100, outside surface 14B of second member 14 and inside surface 10A of first member 10 may not always be in contact with each other at predisposed contact locations 18. For example, predisposed contact locations 18 may provide locations of closer proximity between outside surface 14B of second member 14 and inside surface 10A of first member 10 so that during relative movement, contact between outside surface 14B and inside surface 10A would more likely be established at predisposed contact locations 18 so as to provide support between first member 10 and second member 14. Depending on the configuration of medical instrument 100 and on the type of actuation taking place between first member 10 and second member 14, the presence of predisposed contact locations 18 may require less relative movement (i.e., backlash) for first member 10 and second member 14 to position themselves relative to each other in a position suitable for transferring an actuation force along either first member 10 or second member 14.

As mentioned above, first member 10 and/or second member 14 may comprise a substantially rigid or, alternatively, a flexible material permitting medical instrument 100 to resiliently bend to accommodate the anatomy of a patient. For example, in some embodiments, first member 10 and second member 14 may be deformed to follow a tortuous path during normal use. Accordingly, such flexible material may permit first member 10 and second member 14 to conform to such tortuous path without exhibiting significant plastic deformation. However, whether or not the material(s) used for first member 10 and second member 14 is/are considered flexible or rigid, predisposed contact locations 18 are intended to be permanently formed by first member 10 and/or second member 14 so as to remain defined during normal use of medical instrument 10. For example, predisposed contact locations 18 are intended to remain at least partially permanently defined even though first member 10 and second member 14 may be non-permanently (e.g., elastically, resiliently) bent to follow a tortuous path. Accordingly, permanent predisposed contact locations 18 may be able to at least partially withstand temporary (i.e., elastic, resilient) deformation that first member 10 and/or second member 14 may be designed to undergo during normal use.

Predisposed contact locations 18 are intended to provide locations of mutual support between first member 10 and/or second member 14 during relative longitudinal movement therebetween and during normal use of medical instrument 100. Accordingly, there may not necessarily be contact at predisposed contact locations 18 when medical instrument 100 is at rest. For example, first member 10 and/or second member 14 may be permanently formed so that there is no contact at some or all of predisposed contact locations 18 and that a clearance is instead provided between first member 10 and second member 14 at predisposed contact locations 18 when medical instrument 100 is at rest. Alternatively, first member 10 and/or second member 14 may be permanently formed so that there is contact at some or all of predisposed contact locations 18 between first member 10 and second member 14 when medical instrument 100 is at rest. Furthermore, in some embodiments, first member 10 and/or second member 14 may be permanently formed so that there is an interference (i.e., friction) fit between first member 10 and second member 14 so as to produce a biasing force between first member 10 and second member 14 at predisposed contact locations 18 when medical instrument 100 is at rest.

First member 10 and/or second member 14 may undergo longitudinal pushing and/or pulling forces to cause relative movement between first member 10 and second member 14 and it is understood that such forces may cause some non-permanent deformation (e.g., compression, elongation, bending, torsion) of first member 10 and/or second member 14 during normal operation of medical instrument 100. However, predisposed contact locations 18 defined via permanent forming of first member 10 and/or second member 14 are intended to at least partially withstand such non-permanent deformation that may be experienced by first member 10 and/or second member 14 during normal use.

As shown in FIG. 1A, second member 14 may be permanently formed so that at least part of center line 14C has a sinuous shape while lumen 12 has a substantially straight center line 12C. Such deviations between center line 14C and center line 12C may cause predisposed contact locations 18 to be defined by one or more peaks defined by second member 14 due to the sinuous shape of center line 14C. For example, second member 14 may have a sinuous portion having a peak to peak dimension (i.e., amplitude) substantially equal to a diameter of lumen 12.

As shown in the subsequent embodiments, predisposed contact locations 18 could instead be defined by way of at least part of center line 12C having a sinuous shape instead of or in addition to center line 14C. The term “sinuous” as used herein is intended to encompass shapes that include curves, bends and turns and also combinations of straight lines with curves, bends, turns. For example, such sinuous shapes could include one or more of wavy, sinusoidal, helical, saw-tooth, square wave, winding, corrugated and undulated shapes. Even though the longitudinal waviness of some of the components shown herein is represented by a sine wave, the waviness may be of other type(s) such as rectangular, triangular, trapezoid, semi-circular, semi-ellipsoid, or combination(s) thereof. Also the waviness in the components shown herein may be periodic, non-periodic, planar and/or multi-planar.

Accordingly, part of center line 14C and/or part of center line 12C may have one or more sinuous portions. Such sinuous portions may in some embodiments consequently cause outside surface 14B of second member 14 and/or inside surface 10A of first member 10 to have one or more sinuous portions in order to define predisposed contact portions 18. With respect to the embodiment of FIGS. 1A-1D, a sinuous portion of center line 14C may extend along an entire length of first member 10 or along only a portion of the length of first member 10.

The shape(s) of inside surface 10A of first member 10 and/or of outside surface 14B of second member 14 may be configured to provide a plurality of predisposed contact locations 18 that are interspaced along lumen 12. For example predisposed contact locations 18 may be separated by regions that are free of such predisposed contact locations 18. The spacing between predisposed contact locations 18 as well as the size and number of predisposed contact locations 18 may be selected based on geometric factors, operating parameters as well as on the mechanical properties (e.g., stiffness) of first member 10 and/or second member 14. For example, the spacing between predisposed contact locations 18 may be selected based on the susceptibility of second member 14 to deform (e.g., buckle) under compressive forces and also based on the outer diameter of second member 14 relative to the diameter of lumen 12.

Alternatively, predisposed contact locations 18 may be joined together so as to form a continuous line or surface area for interfacing between inside surface 10A of first member 10 and outside surface 14B of second member 14. For example, in case of second member 14 and center line 14C having a helical shape, predisposed contact locations 18 could comprise a continuous contact line extending helically along which contact between inside surface 10A of first member 10 and outside surface 14B of second member 14 may occur. Also, depending on the configuration of first member 10 and second member 14, predisposed contact locations 18 could include one or more points, one or more lines (e.g., linear, arcuate) and/or one or more surface areas.

The formation of predisposed contact portions 18 by way of sinuous center line 14C and/or sinuous center line 12C may require no additional obstructions such as fins or protrusions that extend from first member 10 and/or second member 14 and that may obstruct flow in passage 16. Accordingly, even though some of the embodiments disclosed herein may cause some additional resistance to fluid flow in passage 16, such flow resistance may be significantly less that would otherwise be encountered with the use of fins or other protrusions.

FIG. 1B shows an elevation view of medical instrument 100 with a cutaway portion where first member 10 and center line 12C are shown as being substantially straight and second member 14 is permanently formed so that center line 14C is sinuous. The illustration shown in FIG. 1B represents a state where medical instrument 100 is not bent and may be at rest.

FIG. 1C shows a side view of medical instrument 100 with another cutaway portion where first member 10 and center line 12C are shown as being bent and second member 14 is permanently formed so that center line 14C is sinuous. The illustration shown in FIG. 1C may represent a state where medical instrument 100 is bent to conform to the anatomy of a patient during use (e.g., during a flexible endoscopy procedure). FIG. 1C shows that predisposed contact location 18 are maintained during bending of medical instrument 100 during normal use.

FIG. 1D shows a cross-sectional view of medical instrument 100 taken along line 1-1 shown in FIG. 1C. Line 1-1 in FIG. 1D is positioned at one of the predetermined contact locations 18. FIG. 1D shows the substantially circular cross-section profile of lumen 12 and also of second member 14.

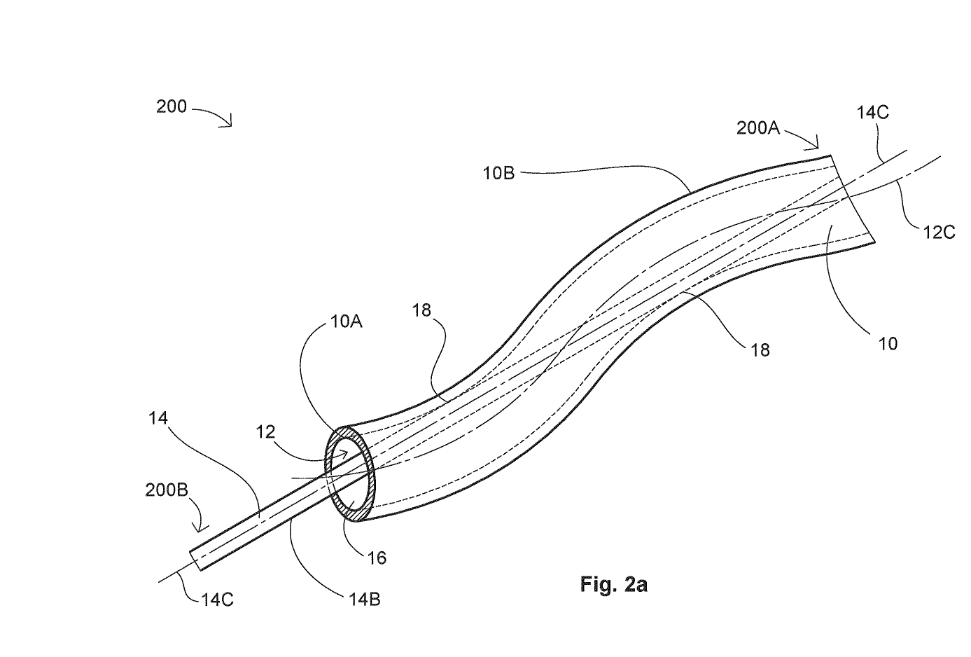

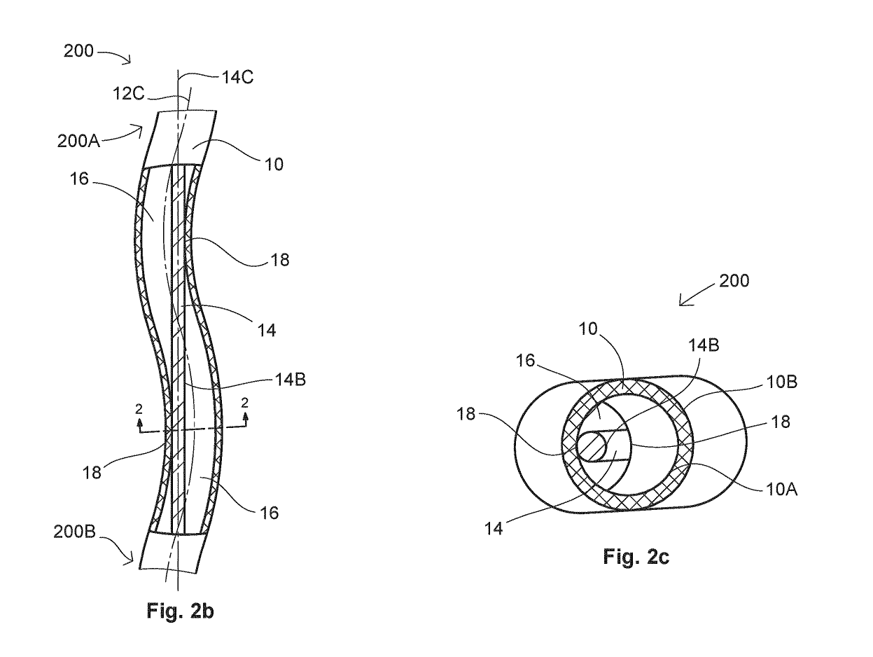

FIGS. 2A-2C illustrate different views of an exemplary medical instrument 200 according to another embodiment of the present disclosure. FIG. 2A shows a perspective view of medical instrument 200, which may comprise a catheter or an endoscopic system for example. Medical instrument 200 may have proximal portion 200A that may be associated with one or more actuation means (not shown) of known or other type(s) and distal portion 200B that may be associated with one or more end effectors (not shown) of known or other type(s). Medical instrument 200 contains elements previously described above in relation to medical instrument 100 so like elements are referenced using like reference numerals and corresponding description is not repeated below.

Medical instrument 200 may comprise first member 10 and second member 14 extending substantially longitudinally in lumen 12. In this embodiment, first member 12 may be permanently formed so that center line 12C or part(s) thereof is/are sinuous so as to define predisposed contact locations 18 between inside surface 10A of first member 10 and outside surface 14B of second member 14 to reduce backlash during relative longitudinal movement between first member 10 and second member 14.

FIG. 2B shows an elevation view of medical instrument 200 with a cutaway portion where first member 10 is permanently formed so that center line 12C is sinuous and second member 14 and center line 14C are substantially straight. The configuration of medical instrument 200 shown in FIG. 2B may correspond to medical instrument 200 being at rest.

FIG. 2C shows a cross-sectional view of medical instrument 200 taken along line 2-2 shown in FIG. 2B. Line 2-2 in FIG. 2C is positioned at one of the predetermined contact locations 18. FIG. 2C shows the substantially circular cross-section profile of lumen 12 and of second member 14.

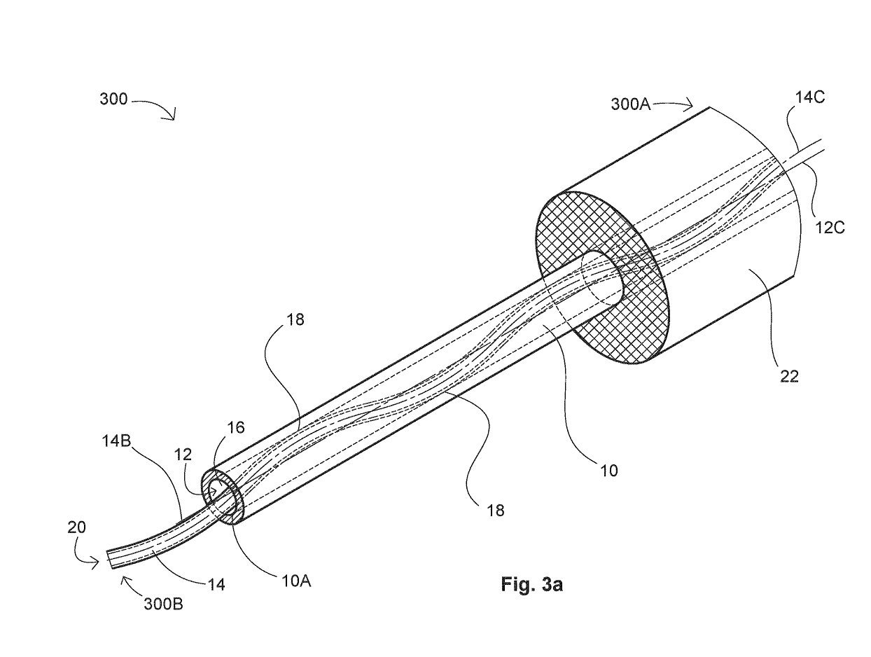

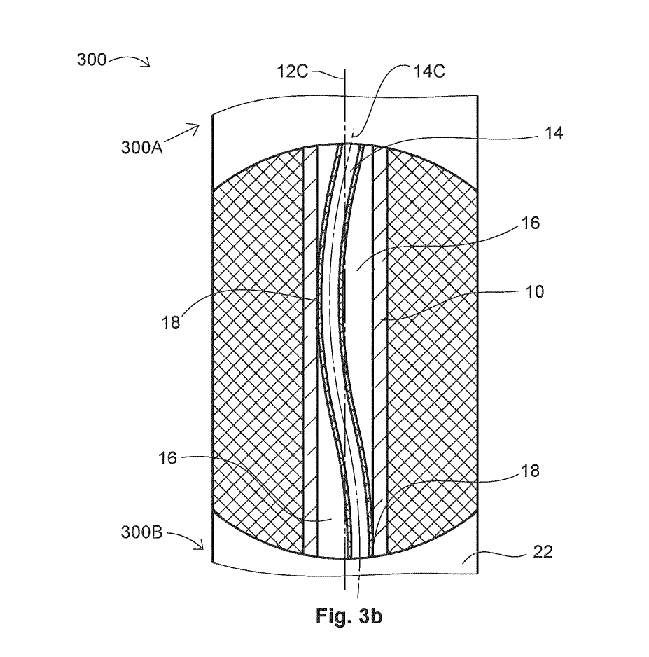

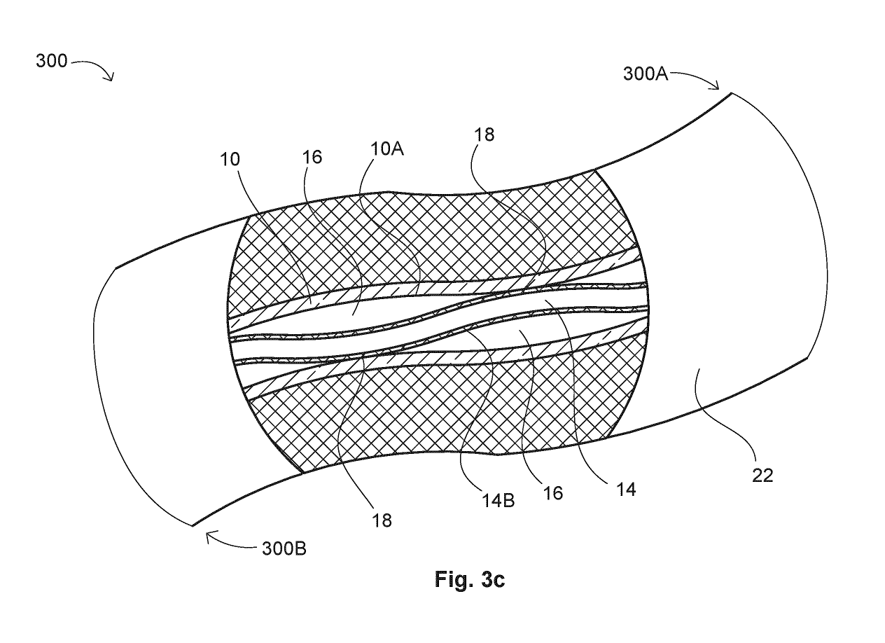

FIGS. 3A-3C illustrate different views of an exemplary medical instrument 300 according to another embodiment of the present disclosure. FIG. 3A shows a perspective view of medical instrument 300, which may comprise a catheter or an endoscopic system for example. Medical instrument 300 may have proximal portion 300A that may be associated with one or more actuation means (not shown) of known or other type(s) and distal portion 300B that may be associated with one or more end effectors (not shown) of known or other type(s). Medical instrument 300 contains elements previously described above in relation to other embodiments so like elements are referenced using like reference numerals and corresponding description is not repeated below.

Medical instrument 300 may comprise first member 10 and second member 14 extending substantially longitudinally in lumen 12 of first member 10. In this embodiment, second member 14 may have a tubular configuration and may have its own lumen 20. Second member 14 may be permanently formed so that at least part of center line 14C is sinuous so as to define predisposed contact locations 18 between inside surface 10A of first member 10 and outside surface 14B of second member 14 to reduce backlash during relative longitudinal movement between first member 10 and second member 14.

Medical instrument 300 may comprise insertion tube 22, which is schematically shown in FIGS. 3A-3C. Other associated components such as electrical cables, fibre optic bundles and control wires are not shown for the sake of clarity.

FIG. 3B shows an elevation view of medical instrument 300 with a cutaway portion where center line 12C is substantially straight and second member 14 is permanently formed so that center line 14C is sinuous. The configuration of medical instrument 300 shown in FIG. 3B may correspond to medical instrument 300 being at rest.

FIG. 3C shows a side view of medical instrument 300 with another cutaway portion where first member 10 and center line 12C are shown as being bent and second member 14 is permanently formed so that center line 14C is sinuous. The illustration shown in FIG. 3C may represent a state where medical instrument 300 is bent to conform to the anatomy of a patient during use (e.g., during a flexible endoscopy procedure). FIG. 3C shows that predisposed contact locations 18 are maintained during bending of medical instrument 300 during normal use.

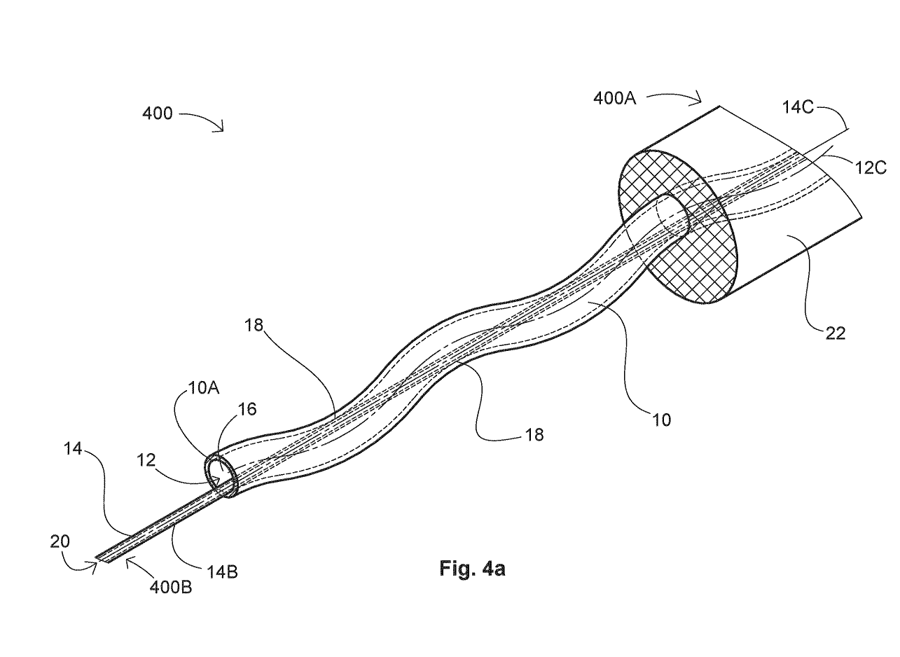

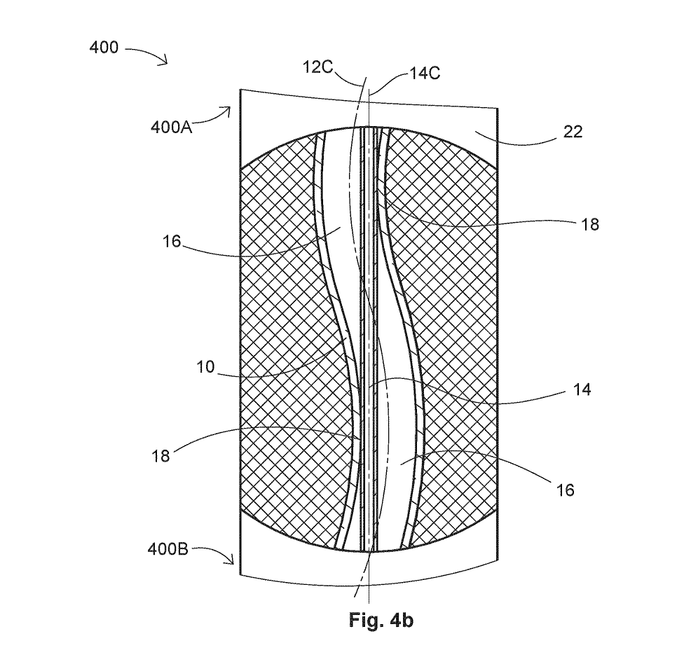

FIGS. 4A and 4B illustrate different views of an exemplary medical instrument 400 according to another embodiment of the present disclosure. FIG. 4A shows a perspective view of medical instrument 400, which may comprise a catheter or an endoscopic system for example. Medical instrument 400 may have proximal portion 400A that may be associated with one or more actuation means (not shown) of known or other type(s) and distal portion 400B that may be associated with one or more end effectors (not shown) of known or other type(s). Medical instrument 400 contains elements previously described above in relation to other embodiments so like elements are referenced using like reference numerals and corresponding description is not repeated below.

Medical instrument 400 may comprise first member 10 and second member 14 extending substantially longitudinally in lumen 12 of first member 10. In this embodiment, second member 14 may have a tubular configuration and may have its own lumen 20. First member 10 may be permanently formed so that at least part of center line 12C is sinuous so as to define predisposed contact locations 18 between inside surface 10A of first member 10 and outside surface 14B of second member 14 to reduce backlash during relative longitudinal movement between first member 10 and second member 14. Medical instrument 400 may comprise insertion tube 22, which is schematically shown in FIGS. 4A and 4B. Other associated components such as electrical cables, fibre optic bundles and control wires are not shown for the sake of clarity.

FIG. 4B shows an elevation view of medical instrument 400 with a cutaway portion where first member 10 is permanently formed so that center line 12C is sinuous and second member 14 and center line 14C are substantially straight. The configuration of medical instrument 400 shown in FIG. 4B may correspond to medical instrument 400 being at rest.

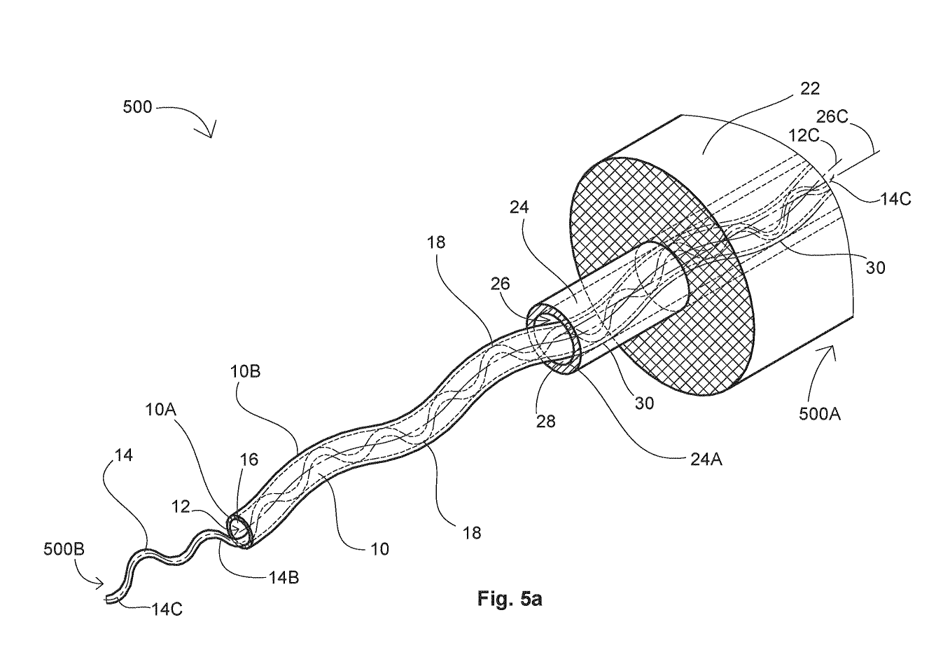

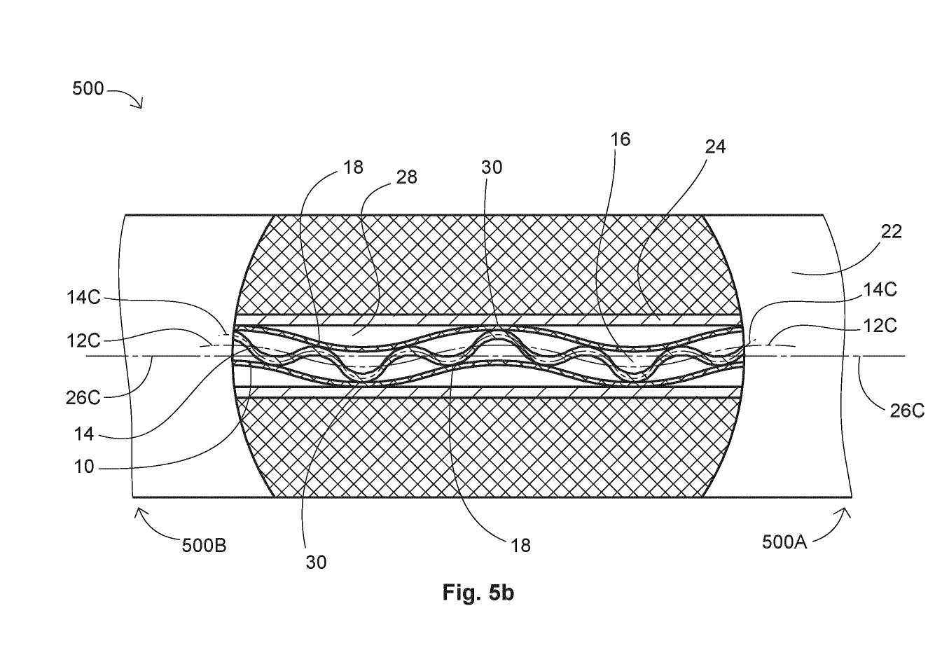

FIGS. 5A and 5B illustrate different views of an exemplary medical instrument 500 according to another embodiment of the present disclosure. FIG. 5A shows a perspective view of medical instrument 500, which may comprise a catheter or an endoscopic system for example. Medical instrument 500 may have proximal portion 500A that may be associated with one or more actuation means (not shown) of known or other type(s) and distal portion 500B that may be associated with one or more end effectors (not shown) of known or other type(s). Medical instrument 500 contains elements previously described above in relation to other embodiments so like elements are referenced using like reference numerals and corresponding description is not repeated below.

Medical instrument 500 may comprise first member 10 and second member 14 extending substantially longitudinally in lumen 12 of first member 10. First member 10 may be permanently formed so that at least part of center line 12C is sinuous and second member 14 may also be permanently formed so that at least part of center line 14C is also sinuous. However, center line 12C and center line 14C may comprise be non-parallel portions. For example, center line 12C and center line 14C may comprise sinusoidal portions that have different periods so as to define predisposed contact locations 18 between inside surface 10A of first member 10 and outside surface 14B of second member 14 to reduce backlash during relative longitudinal movement between first member 10 and second member 14. Medical instrument 500 may comprise insertion tube 22, which is schematically shown in FIGS. 5A and 5B. Other associated components such as electrical cables, fibre optic bundles and control wires are not shown for the sake of clarity.

Medical instrument 500 may comprising third elongate tubular member 24 having inside surface 24A defining lumen 26 having center line 26C. The characteristics and relationships between third member 24 and first member 10 may be similar to those between first member 10 and second member 14 described above in relation to other embodiments. For example, first member 10 may extend substantially longitudinally in lumen 26 defined by third member 24. First member 10 may extend entirely or only partially in lumen 26. Also, outside surface 10B of first member 10 may cooperate with inside surface 24A of third member 24 to define intermediate longitudinal passage 28 therebetween extending in lumen 26. First member 10 and/or third member 24 may be permanently formed to define predisposed contact locations 30 between inside surface 24A of third member 24 and outside surface 10B of first member 10 to reduce backlash during relative longitudinal movement between first member 10 and third member 24. Passage 28 may be configured to permit the passage of fluid though lumen 26 during irrigation or suction of a treatment site for example. The formation of predisposed contact portions 30 by way of sinuous center line 26C and/or sinuous center line 12C may require no additional obstructions such as fins or protrusions that extend from first member 10 and/or third member 24 and that may obstruct flow in passage 28.

In some embodiments, inside surface 10A of first member 10 and outside surface 10B of first member 10 may be substantially parallel and first member 10 and third member 24 may be permanently formed to have non-parallel portions between center line 12C and center line 26C. In some embodiments, at least part of one of the center line 12C and center line 26C may be sinuous. For example, inside surface 24A of third member 24 may be permanently formed so that at least part of center line 26C is substantially straight while at least part of center line 12C may be sinuous.

Lumen 26 may have a substantially uniform cross-sectional profile along center line 26C where the cross-sectional profile of lumen 26 is substantially perpendicular to center line 26C. Cross-sectional profile of lumen 26 may be substantially circular and provide the advantages described above.

FIG. 5B shows a side view of medical instrument 500 with a cutaway portion where first member 10 is permanently formed so that center line 12C is sinuous, second member 14 is permanently formed so that center line 14C is sinuous and center line 26C of lumen 26 is substantially straight. The configuration of medical instrument 500 shown in FIG. 5B may correspond to medical instrument 500 being at rest.

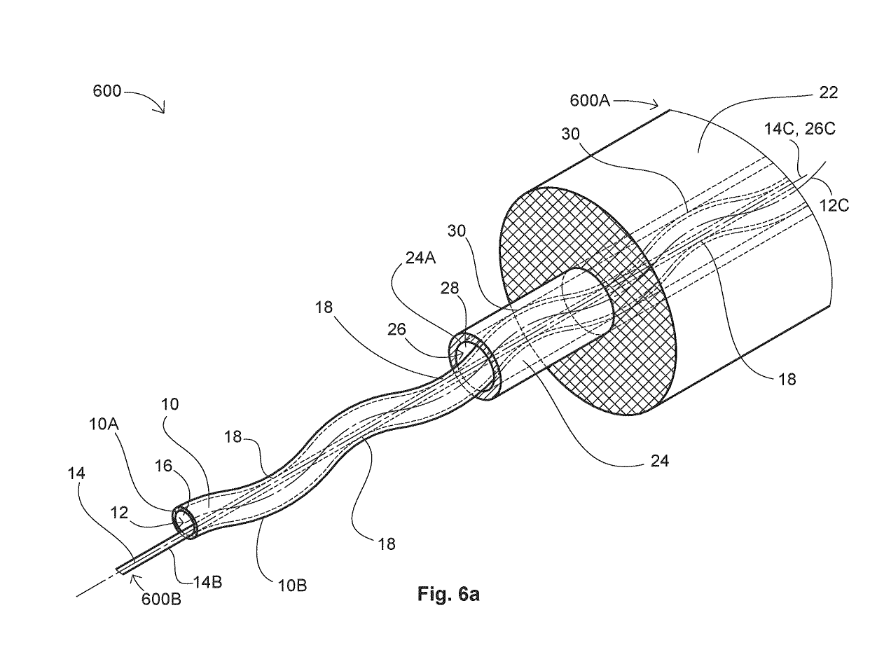

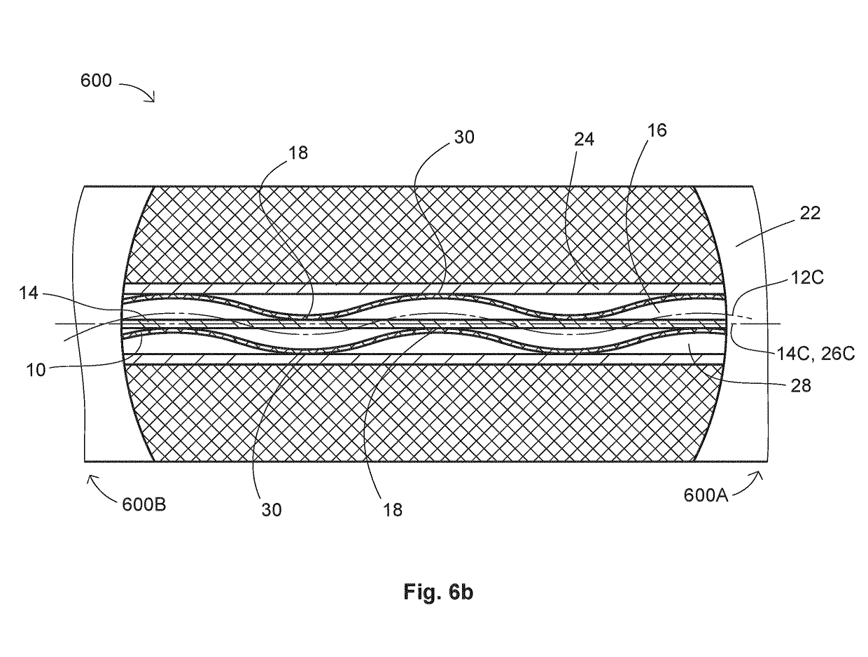

FIGS. 6A and 6B illustrate different views of an exemplary medical instrument 600 according to another embodiment of the present disclosure. FIG. 6A shows a perspective view of medical instrument 600, which may comprise a catheter or an endoscopic system for example. FIG. 6B shows a side view of medical instrument 600 with a cutaway portion. Medical instrument 600 may have proximal portion 600A that may be associated with one or more actuation means (not shown) of known or other type(s) and distal portion 600B that may be associated with one or more end effectors (not shown) of known or other type(s). Medical instrument 600 contains elements previously described above in relation to other embodiments so like elements are referenced using like reference numerals and corresponding description is not repeated below. Medical instrument 600 may comprise insertion tube 22, which is schematically shown in FIGS. 6A and 6B. Other associated components such as electrical cables, fibre optic bundles and control wires are not shown for the sake of clarity.

Similarly to medical instrument 500, medical instrument 600 may comprise first member 10, second member 14 and third member 24. First member 10 may be permanently formed so that at least part of center line 12C is sinuous so as to define predisposed contact locations 18 between first member 10 and second member 14 and also define predisposed contact locations 30 between first member 10 and third member 24. In this particular embodiment, both center line 14C and center line 26C are shown as being substantially straight.

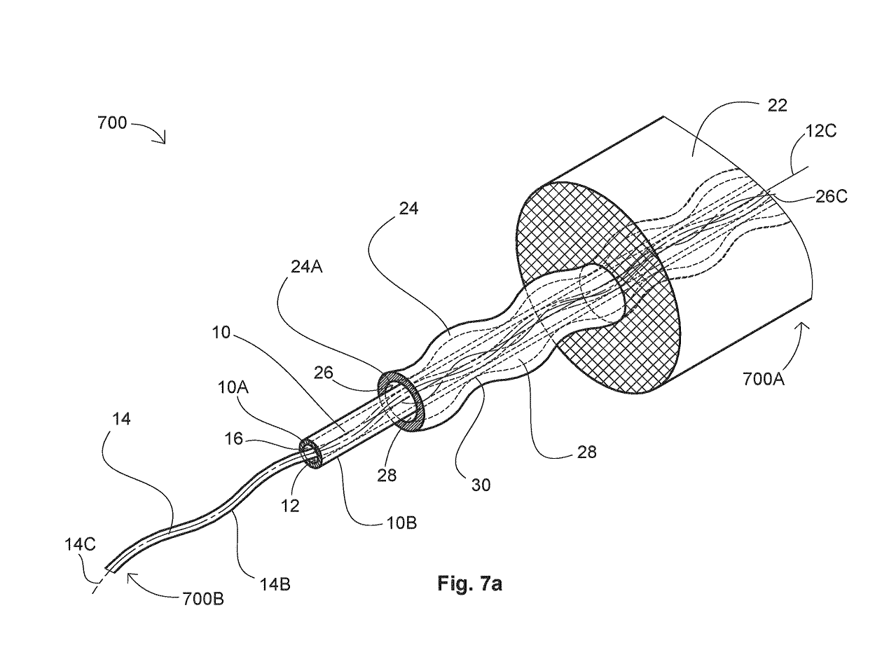

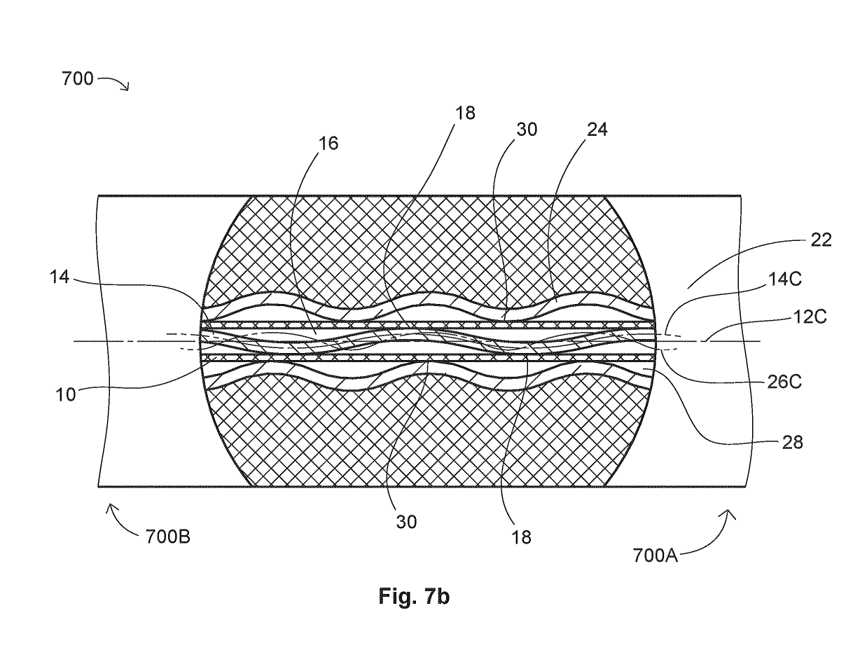

FIGS. 7A and 7B illustrate different views of an exemplary medical instrument 700 according to another embodiment of the present disclosure. FIG. 7A shows a perspective view of medical instrument 700, which may comprise a catheter or an endoscopic system for example. FIG. 7B shows a side view of medical instrument 700 with a cutaway portion. Medical instrument 700 may have proximal portion 700A that may be associated with one or more actuation means (not shown) of known or other type(s) and distal portion 700B that may be associated with one or more end effectors (not shown) of known or other type(s). Medical instrument 700 contains elements previously described above in relation to other embodiments so like elements are referenced using like reference numerals and corresponding description is not repeated below. Medical instrument 700 may comprise insertion tube 22, which is schematically shown in FIGS. 7A and 7B. Other associated components such as electrical cables, fibre optic bundles and control wires are not shown for the sake of clarity.

Similarly to medical instruments 500 and 600, medical instrument 700 may comprise first member 10, second member 14 and third member 24. Second member 14 may be permanently formed so that at least part of center line 14C is sinuous so as to define predisposed contact locations 18 between first member 10 and second member 14. Third member 24 may be permanently formed so that at least part of center line 26C of lumen 26 is sinuous so as to define predisposed contact locations 30 between first member 10 and third member 24. In this particular embodiment, center line 12C of lumen 12 is shown as being substantially straight.

Described and illustrated herein are several embodiments of medical instruments (e.g., endoscopic instruments and endoscopic systems) having reduced or eliminated backlash during operation. The embodiments described herein do not make use of protrusions or fins that would obstruct flow through fluid passages 16 and 28. Also, the embodiments may provide for convenient seal and attachment of the medical instruments to other devices, accessories and components.

It should be noted that the medical instruments described herein are not limited to work with endoscopes, but could also be used as standalone catheters or parts of catheter systems not requiring an endoscope.

Various aspects of the present disclosure could also be equally applied to multi-lumen instruments and systems. Various aspects of the present disclosure could also be equally applied to the field of industrial endoscopy.

The permanent forming of predisposed contact locations 18, 30 may be achieved during manufacturing so that the components are permanently formed to substantially retain the desired shapes. Alternatively, permanent forming of a component may also include situations where the component retains its desired shape during the relevant use (e.g., reducing backlash during relative movement) of the medical instrument but does not necessarily retains its shape at other times. For example, first member 10, second member 14 and/or third member 24 could comprise flexible or malleable material(s) where the “permanent” sinuous shape may be induced by way of another, more rigid, shaping member inserted therein for example. In some embodiments, first member 10, second member 14 and/or third member 24 could, for example, comprise shape memory material(s) where the sinuous shape may be induced by delivery or removal of energy such as a flow of electric current and/or a change in temperature.

### Example 1—Backlash Reduction Measurements

This example presents experimental results that show the reduction in backlash that may be provided using medical instruments according to the present disclosure. For the purpose of the measurements a wavy inner member (i.e., second member 14) was used in conjunction with a straight tubular outer member (i.e., first member 10). To illustrate and quantify the benefits of backlash reduction associated with the movement of the wavy inner member (wire) inside the straight outer member (tube), the following parameters presented in Table 1 were used. In order to simulate the tortuous anatomy of a patient, the tubes (with the wire inside) were wound in two loops of 20 cm diameter at one end.

As expected, the backlash of the baseline straight wires is lower with the larger wire diameters, as they fill the inside of the tubes more. In the extreme case where the outside diameter of the inner wire would be the same as the inside diameter of the tube, the backlash would in theory be zero.

The tables 2-5 below show a significant backlash reduction with the wavy wires in comparison with the straight wires. The highest reduction observed occurred when the peak to peak (PP) value of the wavy wire was similar to the inside diameter of the tube. Furthermore, the lower the period of the waves, which means more contact areas between the tubes and the wires, the greater the backlash reduction.

TABLE 1
Experimental parameters associated
with the inner and outer members.

| Parameters | Values |
|---|---|
| inner members | Material: |
| | Stainless steel wires with a tensile strength |
| | above 300 ksi (2068 MPa). |
| | Outside Diameters: |
| | 0.020″ (0.508 mm) |
| | 0.025″ (0.635 mm) |
| | Length: |
| | 180 cm |
| | Shapes: |
| | straight |
| | sinuous with peak to peak (PP) amplitudes |
| | of 0.052″ (1.32 mm) and 0.126″ (3.20 mm) |
| | and periods of 1″ (25 mm) and 2″ (50 mm) |
| tubular outer | Inside Diameters (ID) and Materials: |
| members | 0.053″ (1.35 mm) - Acetal copolymer |
| | 0.126″ (3.20 mm) - Polytetrafluoroethylene |
| | (PTFE) |

The experimental results of the backlash reduction are tabulated below. The backlash reduction measurements were acquired using minimal force applied to the inner wires relative to the outer tubes so that there was no significant stretching of the wires. The measurements presented in Tables 2-5 do not include the amount of backlash attributed to the elongation of the wire under load. The total amounts of backlash including the elongation of the wires under load are presented in EXAMPLE 3 below.

TABLE 2
Backlash measurements obtained from a baseline straight
wire having a diameter of 0.020″ (0.508 mm) and also
with a wavy wire having a diameter of 0.020″ (0.508
mm), a peak to peak (PP) amplitude of 0.052″ (1.32
mm) and periods of 1″ (25 mm) and 2″ (50 mm).

| | | 0.508 mm Wavy Wire, | | 0.508 mm Wavy Wire, | |
|---|---|---|---|---|---|
| | Baseline | 1.32 mm PP, 50 mm | | 1.32 mm PP, 25 mm | |
| | straight wire | period | | period | |
| Tube ID | Backlash | Backlash | % | Backlash | % |
| (mm) | (mm) | (mm) | Change | (mm) | Change |
| 1.35 | 11 | 5.25 | −52% | 5 | −55% |
| 3.20 | 33 | 29 | −12% | 27.5 | −17% |

TABLE 3
Backlash measurements obtained from a baseline straight
wire having a diameter of 0.020″ (0.508 mm) and also
with a wavy wire having a diameter of 0.020″ (0.508
mm), a peak to peak (PP) amplitude of 0.126″ (3.20
mm) and periods of 1″ (25 mm) and 2″ (50 mm).

| | | 0.508 mm Wavy Wire, | | 0.508 mm Wavy Wire, | |
|---|---|---|---|---|---|
| | Baseline | 3.20 mm PP, 50 mm | | 3.20 mm PP, 25 mm | |
| | straight wire | period | | period | |
| Tube ID | Backlash | Backlash | % | Backlash | % |
| (mm) | (mm) | (mm) | Change | (mm) | Change |
| 1.35 | 11 | — | — | — | — |
| 3.20 | 33 | 12.8 | −61% | 11 | −67% |

TABLE 4
Backlash measurements obtained from a baseline straight
wire having a diameter of 0.025″ (0.635 mm) and also
with a wavy wire having a diameter of 0.025″ (0.635
mm), a peak to peak (PP) amplitude of 0.052″ (1.32
mm) and periods of 1″ (25 mm) and 2″ (50 mm).

| | | 0.635 mm Wavy Wire, | | 0.635 mm Wavy Wire, | |
|---|---|---|---|---|---|
| | Baseline | 1.32 mm PP, 50 mm | | 1.32 mm PP, 25 mm | |
| | straight wire | period | | period | |
| Tube ID | Backlash | Backlash | % | Backlash | % |
| (mm) | (mm) | (mm) | Change | (mm) | Change |
| 1.35 | 8.5 | 4.25 | −50% | 2 | −76% |
| 3.20 | 28 | 22 | −21% | 20.25 | −28% |

TABLE 5
Backlash measurements obtained from a baseline straight
wire having a diameter of 0.025″ (0.635 mm) and also
with a wavy wire having a diameter of 0.025″ (0.635
mm), a peak to peak (PP) amplitude of 0.126″ (3.20
mm) and periods of 1″ (25 mm) and 2″ (50 mm).

| | | 0.635 mm Wavy Wire, | | 0.635 mm Wavy Wire, | |
|---|---|---|---|---|---|
| | Baseline | 3.20 mm PP, 50 mm | | 3.20 mm PP, 25 mm | |
| | straight wire | period | | period | |
| Tube ID | Backlash | Backlash | % | Backlash | % |
| (mm) | (mm) | (mm) | Change | (mm) | Change |
| 1.35 | 8.5 | — | — | — | — |
| 3.20 | 28 | 5.75 | −79% | 2.5 | −91% |

### Example 2—Elongation Measurements

This example presents experimental results that show amounts of elongation measured in the inner member (wire) in straight and wavy configurations under the application of forces typically encountered during operation of medical devices as disclosed in the present disclosure. In some cases, typical forces required in manipulation or actuation of endoscopic devices is less than 2 lbs (8.9 N). The results presented in Tables 6-9 provide an indication of how much of the backlash measured (see Examples 1 and 3) may be attributed to the elongation of the wire. As expected, the wavy wires exhibited a higher elongation than the straight wires under the application of the same longitudinal tensile force. The wavy wires used in this example were in accordance with the parameters specified in Table 1.

TABLE 6
Elongation measurements from a baseline straight wire
having a diameter of 0.020″ (0.508 mm) and also with
a wavy wire having a diameter of 0.020″ (0.508 mm),
a peak to peak (PP) amplitude of 0.052″ (1.32 mm),
periods of 1″ (25 mm) and 2″ (50 mm) and a length of 180 cm.

| | Baseline | 0.508 mm Wavy Wire, | 0.508 mm Wavy Wire, |
|---|---|---|---|
| Load | straight Wire | 1.32 mm PP, 50 mm | 1.32 mm PP, 25 mm |
| (Lbs) | (mm) | period (mm) | period (mm) |
| 1 | 0.15 | 1.2 | 1.2 |
| 2 | 0.39 | 1.69 | 2.21 |
| 3 | 0.61 | 2.14 | 3.14 |
| 5 | 1.18 | 3.11 | 4.27 |
| 10 | 2.67 | 4.97 | 6.72 |

TABLE 7
Elongation measurements from a baseline straight wire
having a diameter of 0.020″ (0.508 mm) and also with
a wavy wire having a diameter of 0.020″ (0.508 mm),
a peak to peak (PP) amplitude of 0.126″ (3.20 mm),
periods of 1″ (25 mm) and 2″ (50 mm) and a length of 180 cm.

| | Baseline | 0.508 mm Wavy Wire, | 0.508 mm Wavy Wire, |
|---|---|---|---|
| Load | straight Wire | 3.20 mm PP, 50 mm | 3.20 mm PP, 25 mm |
| (Lbs) | (mm) | period (mm) | period (mm) |
| 1 | 0.15 | 5.25 | 9.16 |
| 2 | 0.39 | 7.73 | 15.14 |
| 3 | 0.61 | 8.71 | 20.03 |
| 5 | 1.18 | 10.13 | 25.83 |
| 10 | 2.67 | 12.39 | 32.71 |

TABLE 8
Elongation measurements from a baseline straight wire
having a diameter of 0.025″ (0.635 mm) and also with
a wavy wire having a diameter of 0.025″ (0.635 mm),
a peak to peak (PP) amplitude of 0.052″ (1.32 mm),
periods of 1″ (25 mm) and 2″ (50 mm) and a length of 180 cm.

| | Baseline | 0.635 mm Wavy Wire, | 0.635 mm Wavy Wire, |
|---|---|---|---|
| Load | straight Wire | 1.32 mm PP, 50 mm | 1.32 mm PP, 25 mm |
| (Lbs) | (mm) | period (mm) | period (mm) |
| 1 | 0.6 | 0.51 | 0.62 |
| 2 | 0.77 | 0.93 | 1.12 |
| 3 | 0.97 | 1.2 | 1.6 |
| 5 | 1.43 | 1.78 | 2.47 |

TABLE 9
Elongation measurements from a baseline straight wire
having a diameter of 0.025″ (0.635 mm) and also with
a wavy wire having a diameter of 0.025″ (0.635 mm),
a peak to peak (PP) amplitude of 0.126″ (3.20 mm),
periods of 1″ (25 mm) and 2″ (50 mm) and a length of 180 cm.

| | Baseline | 0.635 mm Wavy Wire, | 0.635 mm Wavy Wire, |
|---|---|---|---|
| Load | straight wire | 3.20 mm PP, 50 mm | 3.20 mm PP, 25 mm |
| (Lbs) | (mm) | period (mm) | period (mm) |
| 1 | 0.6 | 3.4 | 5.12 |
| 2 | 0.77 | 5.5 | 9.09 |
| 3 | 0.97 | 7.09 | 12.75 |
| 5 | 1.43 | 8.82 | 19.72 |

### Example 3—Backlash and Elongation

This example presents calculated amounts of combined elongation and backlash measured on the inner member (wire) in straight and wavy configurations under the application of two lbs of force. Tables 10-13 are essentially a calculated combination of measurements from EXAMPLES 1 and 2 above. The wavy inner wires and outer tubes used in this example were in accordance with the parameters specified in Table 1.

It was observed that the reduction in backlash was lower for a given setup when the diameter of the wavy wire is smaller. This is believed to be a result of the wavy wires stretching during the experiments and the stretching contributing to the backlash. The smaller wire diameter resulted in more elongation than the larger wire diameter under the same applied force.

TABLE 10
Backlash + elongation calculations of a baseline straight
wire having a diameter of 0.020″ (0.508 mm) and also
with a wavy wire having a diameter of 0.020″ (0.508 mm),
a peak to peak (PP) amplitude of 0.052″ (1.32 mm), periods
of 1″ (25 mm) and 2″ (50 mm) and a length of 180
cm using an actuation force of 2 lbs (8.9 N).

| | | 0.508 mm Wavy Wire, | | 0.508 mm Wavy Wire, | |
|---|---|---|---|---|---|
| | | 1.32 mm PP, 50 mm | | 1.32 mm PP, 25 mm | |
| | Baseline | period | | period | |
| Tube ID | straight wire | Backlash | % | Backlash | % |
| (mm) | (mm) | (mm) | Change | (mm) | Change |
| 1.35 | 11.39 | 6.94 | −39% | 7.21 | −37% |
| 3.20 | 33.39 | 30.69 | −8% | 29.71 | −11% |

TABLE 11
Backlash + elongation calculations from a baseline straight
wire having a diameter of 0.020″ (0.508 mm) and also
with a wavy wire having a diameter of 0.020″ (0.508 mm),
a peak to peak (PP) amplitude of 0.126″ (3.20 mm), periods
of 1″ (25 mm) and 2″ (50 mm) and a length of 180
cm using an actuation force of 2 lbs (8.9 N).

| | | 0.508 mm Wavy Wire, | | 0.508 mm Wavy Wire, | |
|---|---|---|---|---|---|
| | | 3.20 mm PP, 50 mm | | 3.20 mm PP, 25 mm | |
| | Baseline | period | | period | |
| Tube ID | straight wire | Backlash | % | Backlash | % |
| (mm) | (mm) | (mm) | Change | (mm) | Change |
| 1.35 | 11.39 | — | — | — | — |
| 3.20 | 33.39 | 20.53 | −39% | 26.14 | −22% |

TABLE 12
Backlash + elongation calculations from a baseline straight
wire having a diameter of 0.025″ (0.635 mm) and also
with a wavy wire having a diameter of 0.025″ (0.635 mm),
a peak to peak (PP) amplitude of 0.052″ (1.32 mm), periods
of 1″ (25 mm) and 2″ (50 mm) and a length of 180
cm using an actuation force of 2 lbs (8.9 N).

| | | 0.635 mm Wavy Wire, | | 0.635 mm Wavy Wire, | |
|---|---|---|---|---|---|
| | | 1.32 mm PP, 50 mm | | 1.32 mm PP, 25 mm | |
| | Baseline | period | | period | |
| Tube ID | straight wire | Backlash | % | Backlash | % |
| (mm) | (mm) | (mm) | Change | (mm) | Change |
| 1.35 | 9.27 | 5.18 | −44% | 3.12 | −66% |
| 3.20 | 28.77 | 22.93 | −20% | 21.37 | −26% |

TABLE 13
Backlash + elongation calculations from a baseline straight
wire having a diameter of 0.025″ (0.635 mm) and also
with a wavy wire having a diameter of 0.025″ (0.635 mm),
a peak to peak (PP) amplitude of 0.126″ (3.20 mm), periods
of 1″ (25 mm) and 2″ (50 mm) and a length of 180
cm using an actuation force of 2 lbs (8.9 N).

| | | 0.635 mm Wavy Wire, | | 0.635 mm Wavy Wire, | |
|---|---|---|---|---|---|
| | | 3.20 mm PP, 50 mm | | 3.20 mm PP, 25 mm | |
| | Baseline | period | | period | |
| Tube ID | straight wire | Backlash | % | Backlash | % |
| (mm) | (mm) | (mm) | Change | (mm) | Change |
| 1.35 | 9.27 | — | — | — | — |
| 3.20 | 28.77 | 11.25 | −61% | 11.59 | −60% |

### Example 4—Liquid Flow Measurements

This example presents experimental results that show the effect of the wavy wire on water flow through the tube (i.e., through passage 16 or 28) in comparison with the presence of a baseline wire of the same diameter but that is straight. This demonstrates the effect of the addition of predisposed contact locations 18 or 30 on the fluid flow through the passage 16 or 18. The wavy inner wires and outer tubes used in this example were in accordance with the parameters specified in Table 1.

Tables 14-16 summarize the flow experiments and show the reduction in water flow under an applied pressure of 3 bar for wavy wires in comparison with a straight wire. As expected, when the ratio of wire outside diameter to tube inside diameter is small, the flow reduction is negligible. Similarly when the period of the waves is lower for a given configuration, the resistance to flow offered by the wire is higher.

TABLE 14
Water flow rate measurements inside tubes containing a baseline
straight wire having a diameter of 0.020″ (0.508 mm) and
also with a wavy wire having a diameter of 0.020″ (0.508
mm), a peak to peak (PP) amplitude of 0.052″ (1.32 mm),
periods of 1″ (25 mm) and 2″ (50 mm) and a length of 180 cm.

| | | Baseline - | 0.508 mm Wavy | | 0.508 mm Wavy | |
|---|---|---|---|---|---|---|
| | | 0.508 mm | Wire, 1.32 mm | | Wire, 1.32 mm | |
| Tube | Control | straight | PP, 50 mm period | | PP, 25 mm period | |
| ID | (no wire) | wire | | % | | % |
| (mm) | (ml/min) | (ml/min) | ml/min | Change | ml/min | Change |
| 0.053″ | 286.8 | 189.8 | 177.4 | −7% | 159.4 | −16% |

TABLE 15
Water flow rate measurements inside tubes containing a baseline
straight wire having a diameter of 0.025″ (0.635 mm) and
also with a wavy wire having a diameter of 0.025″ (0.635
mm), a peak to peak (PP) amplitude of 0.052″ (1.32 mm),
periods of 1″ (25 mm) and 2″ (50 mm) and a length of 180 cm.

| | | Baseline - | 0.635 mm Wavy | | 0.635 mm Wavy | |
|---|---|---|---|---|---|---|
| | | 0.635 mm | Wire, 1.32 mm | | Wire, 1.32 mm | |
| | Control | straight | PP, 50 mm period | | PP, 25 mm period | |
| Tube | (no wire) | wire | | % | | % |
| ID | (ml/min) | (ml/min) | ml/min | Change | ml/min | Change |
| 1.35 | 286.8 | 164.8 | 145.5 | −7% | 127.2 | −23% |
| 3.20 | 1016.4 | 1015.3 | 1015.2 | <1% | 1015.1 | <1% |

TABLE 16
Water flow rate measurements inside tubes containing a baseline
straight wire having a diameter of 0.025″ (0.635 mm) and
also with a wavy wire having a diameter of 0.025″ (0.635
mm), a peak to peak (PP) amplitude of 0.126″ (3.20 mm),
periods of 1″ (25 mm) and 2″ (50 mm) and a length of 180 cm.

| | | Baseline - | 0.635 mm Wavy | | 0.635 mm Wavy | |
|---|---|---|---|---|---|---|
| | | 0.635 mm | Wire, 3.20 mm | | Wire, 3.20 mm | |
| | Control | straight | PP, 50 mm period | | PP, 25 mm period | |
| Tube | (no wire) | wire | | % | | % |
| ID | (ml/min) | (ml/min) | ml/min | Change | ml/min | Change |
| 1.35 | 286.8 | 164.8 | — | — | — | — |
| 3.20 | 1016.4 | 1015.3 | 1009.1 | 1% | 1001.7 | 1% |

The above description is meant to be exemplary only, and one skilled in the relevant arts will recognize that changes may be made to the embodiments described without departing from the scope of the invention disclosed. The present disclosure may be embodied in other specific forms without departing from the subject matter of the claims. Also, one skilled in the relevant arts will appreciate that while the instruments disclosed and shown herein may comprise a specific number of elements/components, the instruments could be modified to include additional or fewer of such elements/components. The present disclosure is also intended to cover and embrace all suitable changes in technology. Modifications which fall within the scope of the present invention will be apparent to those skilled in the art, in light of a review of this disclosure, and such modifications are intended to fall within the appended claims. Also, the scope of the claims should not be limited by the preferred embodiments set forth in the examples, but should be given the broadest interpretation consistent with the description as a whole.

1. A medical instrument comprising:
a first elongate member having an inside surface defining a first lumen having a first center line; and
a second elongate member extending in the first lumen and having an outside surface cooperating with the inside surface of the first member to define a first intermediate longitudinal passage therebetween extending in the first lumen, the second member having a second center line;
wherein the first member and the second member are permanently formed to have non-parallel portions between the first center line and the second center line and define one or more predisposed contact locations between the inside surface of the first member and the outside surface of the second member to reduce backlash during relative longitudinal movement between the first member and the second member.

2. The medical instrument as defined in claim 1, wherein at least part of one of the first center line and the second center line is sinuous.

3. The medical instrument as defined in claim 1, wherein the second member is permanently formed so that at least part of the second center line is sinuous.

4. The medical instrument as defined in claim 1, wherein the first member is permanently formed so that at least part of the first center line is sinuous.

5. The medical instrument as defined in claim 1, wherein the first lumen has a substantially uniform cross-sectional profile along the first center line where the cross-sectional profile of the first lumen is substantially perpendicular to the first center line.

6. The medical instrument as defined in claim 5, wherein the cross-sectional profile of the first lumen is substantially circular.

7. The medical instrument as defined in claim 1, wherein the second member has a substantially uniform cross-sectional profile along the second center line where the cross-sectional profile of the second member is substantially perpendicular to the second center line.

8. The medical instrument as defined in claim 7, wherein the cross-sectional profile of the second member is substantially circular.

9. The medical instrument as defined in claim 1, comprising a third elongate member having an inside surface defining a second lumen having a third center line, wherein:
the first member extends in the second lumen;
an outside surface of the first member cooperates with the inside surface of the third member to define a second intermediate longitudinal passage therebetween extending in the second lumen; and
the first member and the third member are permanently formed to define one or more predisposed contact locations between the inside surface of the third member and the outside surface of the first member to reduce backlash during relative longitudinal movement between the first member and the third member.

10. The medical instrument as defined in claim 9, wherein:
the inside surface of the first member and the outside surface of the first member are substantially parallel; and
the first member and the third member are permanently formed to have non-parallel portions between the first center line and the third center line.

11. The medical instrument as defined in claim 9, wherein at least part of one of the first center line and the third center line is sinuous.

12. The medical instrument as defined in claim 9, wherein the inside surface of the third member is permanently formed so that at least part of the third center line is sinuous.

13. The medical instrument as defined in claim 9, wherein the second lumen has a substantially uniform cross-sectional profile along the third center line where the cross-sectional profile of the second lumen is substantially perpendicular to the third center line.

14. The medical instrument as defined in claim 13, wherein the cross-sectional profile of the second lumen is substantially circular.

15. The medical instrument as defined in claim 1, wherein the predisposed contact locations between the inside surface of the first member and the outside surface of the second member are interspaced along the first lumen.

16. A medical instrument comprising:
a first member having an inside surface defining a first lumen; and
a second member extending in the first lumen and having an outside surface cooperating with the inside surface of the first member to define an intermediate longitudinal passage therebetween extending in the first lumen;
wherein one of the inside surface of the first member and the outside surface of the second member is permanently formed to have a sinuous shape defining one or more predisposed locations for contacting the other one of the inside surface of the first member and the outer surface of the second member to reduce backlash during relative longitudinal movement between the first member and the second member.

17. The medical instrument as defined in claim 16, wherein the predisposed contact locations are interspaced along the first lumen.

18. The medical instrument as defined in claim 16, wherein the outside surface of the second member is permanently formed to have a sinuous shape.

19. The medical instrument as defined in claim 16, wherein the inside surface of the first member is permanently formed to have a sinuous shape.

20. The medical instrument as defined in claim 16, wherein the first lumen has a substantially uniform cross-sectional profile along a center line of the first lumen where the cross-sectional profile of the first lumen is substantially perpendicular to the center line of the first lumen.

21. The medical instrument as defined in claim 20, wherein the cross-sectional profile of the first lumen is substantially circular.

22. The medical instrument as defined in claim 16, wherein the second member has a substantially uniform cross-sectional profile along a center line of the second member where the cross-sectional profile of the second member is substantially perpendicular to the center line of the second member.

23. The medical instrument as defined in claim 22, wherein the cross-sectional profile of the second member is substantially circular.

24. The medical instrument as defined in claim 16, comprising
a third elongate member having an inside surface defining a second lumen, wherein:
the first member extends in the second lumen;
an outside surface of the first member cooperates with the inside surface of the third member to define a second intermediate longitudinal passage therebetween extending in the second lumen; and
one of the first member and the third member is permanently formed to define one or more predisposed locations for contacting the other one of the first member and the third member to reduce backlash during relative longitudinal movement between the first member and the third member.

25. The medical instrument as defined in claim 24, wherein:
the inside surface of the first member and the outside surface of the first member are substantially parallel; and
one of the first member and the third member is permanently formed to have a sinuous shape.

26. The medical instrument as defined in claim 24, wherein the inside surface of the third member is permanently formed to have a sinuous shape.

27. The medical instrument as defined in claim 24, wherein the second lumen has a substantially uniform cross-sectional profile along a center line of the second lumen where the cross-sectional profile of the second lumen is substantially perpendicular to the center line of the second lumen.

28. The medical instrument as defined in claim 27, wherein the cross-sectional profile of the second lumen is substantially circular.

29. A medical instrument comprising:
a first member having an inside surface defining a first lumen having a first center line, the inside surface being defined by a first cross-sectional profile swept along the first center line; and
a second member extending in the first lumen and having an outside surface cooperating with the inside surface of the first member to define a first intermediate longitudinal passage therebetween extending in the first lumen, the second member having a second center line, the outside surface of the second member being defined by a second cross-sectional profile swept along the second center line;
wherein one of the first member and the second member is permanently formed to have a sinuous respective first center line or second center line and define one or more predisposed locations for contacting the other one of the inside surface of the first member and the outer surface of the second member to reduce backlash during relative longitudinal movement between the first member and the second member.

30. The medical instrument as defined in claim 29, wherein the predisposed contact locations are interspaced along the first lumen.

31. The medical instrument as defined in claim 29, wherein the second member is permanently formed so that at least part of the second center line is sinuous.

32. The medical instrument as defined in claim 29, wherein the first member is permanently formed so that at least part of the first center line is sinuous.

33. The medical instrument as defined in claim 29, wherein the cross-sectional profile of the first lumen is substantially perpendicular to the first center line and is substantially circular.

34. The medical instrument as defined in claim 29, wherein the cross-sectional profile of the second member is substantially perpendicular to the second center line and is substantially circular.

35. The medical instrument as defined in claim 29, comprising a third elongate member having an inside surface defining a second lumen having a third center line, wherein:
the first member extends in the second lumen;
an outside surface of the first member cooperates with the inside surface of the third member to define a second intermediate longitudinal passage therebetween extending in the second lumen; and
one of the first member and the third member is permanently formed to define one or more predisposed locations for contacting the other one of the first member and the third member to reduce backlash during relative longitudinal movement between the first member and the third member.

36. The medical instrument as defined in claim 35, wherein:
the inside surface of the first member and the outside surface of the first member are substantially parallel; and
the one of the first member and the third member is permanently formed to have a sinuous respective first center line or third center line.

37. The medical instrument as defined in claim 35, wherein the third member is permanently formed so that at least part of the third center line is sinuous.

38. The medical instrument as defined in claim 35, wherein the second lumen has a substantially uniform cross-sectional profile along the third center line where the cross-sectional profile of the second lumen is substantially perpendicular to the third center line.

39. The medical instrument as defined in claim 38, wherein the cross-sectional profile of the second lumen is substantially circular.

40. The medical instrument as defined in claim 1, wherein the instrument is an endoscopic system.

41. The medical instrument as defined in claim 1, wherein the instrument is a catheter system.

42. The medical instrument as defined in claim 1, wherein the second elongate member is guide wire.

43. The medical instrument as defined in claim 1, wherein the second member has a sinuous portion having a peak to peak dimension substantially equal to a diameter of the first lumen.
